(19) United States
(12) Patent Application Publication
(10) Pub. No.: US 2018/0306741 A1
(43) Pub. Date: Oct. 25, 2018

(54) NANOFIBER YARN BASED ELECTROCHEMICAL SENSOR

(71) Applicant: Lintec of America, Inc., Richardson, TX (US)

(72) Inventors: Takahiro Ueda, Richardson, TX (US); Marcio D. Lima, Richardson, TX (US)

(73) Assignee: Lintec of America, Inc., Richardson, TX (US)

(21) Appl. No.: 15/927,325

(22) Filed: Mar. 21, 2018

Related U.S. Application Data
(60) Provisional application No. 62/487,151, filed on Apr. 19, 2017.

(51) Int. Cl. G01N 27/12; D02G 3/36; D01H 7/92; A61B 5/145; A61B 5/1477

(52) U.S. Cl. CPC G01N 27/127; D02G 3/36; A61B 5/1477; D01H 7/92; A61B 5/14517; G01N 27/126

(57) ABSTRACT

Nanofiber based sensors are described that can be used to detect analytes in biological or non-biological contexts. Each sensor includes at least two nanofiber yarns that are spaced apart from one another so as to avoid electrical (or physical) contact. Each nanofiber yarn of the nanofiber sensor includes a sensing region that is in electrical contact with the rest of the corresponding nanofiber yarn. The sensing regions of the at least two nanofibers are treated with complementary sensing agents so that when the sensing regions (and the corresponding sensing agents) are exposed to the analyte to be detected, an electrical response is detected. This response is then communicated through one or more of the nanofiber yarns for interpretation by a processor.

### CROSS REFERENCE TO RELATED APPLICATIONS

This application claims the benefit of U.S. Provisional Patent Application Ser. No. 62/487,151, filed Apr. 19, 2017, which is hereby incorporated by reference in its entirety

### TECHNICAL FIELD

The present disclosure relates generally to sensors. Specifically, the present disclosure is related to nanofiber yarn based electrochemical sensors.

### BACKGROUND

Biometric sensors are pervasive due in part to the increasing sophistication of microelectrical mechanical sensors (MEMS) and the algorithms used to analyze data produced by these sensors. This pervasiveness is also due in part to the ease with which MEMS are integrated with mobile computing devices via wireless transceivers. The result is that devices that can continuously measure physical movement of a human body (e.g., steps, heart beats, movement during sleep) are common.

Less common are sensors that can measure chemicals produced by a human body. This type of sensor is capable of monitoring a status of a medical condition (e.g., diabetes) or monitoring a general physiological state of a human body. Potentiometric ion selective electrodes (ISE) have been developed that can measure a variety of analytes in human perspiration. For example, various ISEs can measure a presence or concentration in perspiration of any of the following: sodium, potassium, lactate, uric acid, ammonium or pH. The measured value of an analyte can be indicative of a physiological condition or physiological state. Once sensor data is analyzed, the status of the physiological condition can be displayed to the user so that the user producing the analyte may respond appropriately to the physiological condition.

### SUMMARY

An example of the present disclosure includes a sensor including a substrate; an electrically conductive first nanofiber yarn having a first substrate connected portion connected to the substrate and a first exposed portion, the first exposed portion including a first sensing region; a first sensing agent in contact with the first sensing region of the first nanofiber yarn; an electrically conductive reference nanofiber yarn having a second substrate connected portion connected to the substrate and a second exposed portion, the second exposed portion including a reference sensing region; and a second sensing agent in contact with the reference sensing region of the reference nanofiber yarn, where the first sensing agent and the second sensing agent are selected to generate an electrical potential when both the first sensing agent and the second sensing agent are in electrical contact with each other via a first analyte.

In an embodiment, the first substrate connected portion and the second substrate connected portion are embedded in the substrate. In an embodiment, the first substrate connected portion and the second substrate connected portion are adhered to an outer surface of the substrate. In an embodiment, the first sensing agent and the second sensing agent are disposed at a surface of the first nanofiber yarn and the reference nanofiber yarn, respectively. In an embodiment, the first sensing agent and the second sensing agent are disposed at least in part within a first interior and a second interior of the first nanofiber yarn and the reference nanofiber yarn, respectively. In an embodiment, a separation distance between the first sensing region and the reference sensing region is from 0.5 mm to 2 mm. In an embodiment, the first sensing region and the reference sensing region are configured as a double spiral. In an embodiment, the substrate is a reversibly attachable adhesive substrate. In an embodiment, wherein the sensor is integrated into a fabric. In an embodiment, the sensor further including: a processor in electrical communication with the electrically conductive first nanofiber yarn and the electrically reference conductive nanofiber yarn; and a power source connected to the processor and directly connected to the first sensing region and the reference sensing region via a first electrically connective portion of the first nanofiber yarn integral with the first sensing region and a second electrically connective portion of the reference nanofiber yarn integral with the reference sensing region. In an embodiment, the sensor further including an electrically conductive additional nanofiber yarn including: an additional substrate connected portion connected to the substrate; an additional exposed portion including an additional sensing region; and an additional sensing agent different from the first sensing agent and the reference sensing agent, wherein the additional sensing agent is selected to generate an electrical potential when both the additional sensing agent and the second sensing agent are in contact with an additional analyte different from the first analyte.

In an example, a garment including a fabric including a plurality of non-conductive threads; an electrically conductive first nanofiber yarn woven into the fabric with the plurality of non-conductive threads, the first nanofiber yarn having a first sensing region in contact with a first sensing agent; and an electrically conductive reference nanofiber yarn woven into the fabric with the plurality of non-conductive threads and the first nanofiber yarn, the reference nanofiber yarn having a reference sensing region in contact with a second sensing agent, where the first sensing agent and the second sensing agent are selected to generate an electrical potential when both the first sensing agent and the second sensing agent are in electrical contact with each other via an analyte. In an embodiment, where a separation distance between the first sensing region and the reference sensing region is from 0.5 mm to 2 mm. In an embodiment, the plurality of non-conductive threads urge the first sensing region and the reference sensing region into contact with a surface around which the fabric is disposed. In an embodiment, the garment further including at least a power source electrically connected to the first nanofiber yarn and the reference nanofiber yarn. In an embodiment, the electrically conductive first nanofiber yarn further includes a first electrically connective portion integral with the first sensing region and directly connected to the power source; and the electrically conductive reference nanofiber yarn further includes a second electrically connective portion integral with the first sensing region and directly connected to a power source. In an embodiment, the first sensing agent and the second sensing agent are disposed at a surface of the first nanofiber yarn and the reference nanofiber yarn, respectively. In an embodiment, the first sensing agent and the second sensing agent are disposed at least in part within a first interior and a second interior the first nanofiber yarn and the reference nanofiber yarn, respectively. In an embodiment, the garment further including a processor in electrical communication with the first electrically conductive nanofiber yarn and the electrically conductive reference nanofiber yarn.

### BRIEF DESCRIPTION OF THE DRAWINGS

FIG. 1A is a cross-sectional view of a nanofiber yarn electrochemical sensor disposed on perspiring skin, the view taken perpendicular to a longitudinal axis of nanofiber yarns of the sensor, in an embodiment.

FIG. 1B is a plan view of the nanofiber yarn electrochemical sensor of FIG. 1A viewed through the substrate, in an embodiment.

FIG. 2A is a cross-sectional view of a nanofiber yarn electrochemical sensor in which sensing agents are disposed in an interior of nanofiber yarns of the sensor, and the nanofiber yarns are partially embedded in a substrate, the view taken perpendicular to a longitudinal axis of the nanofiber yarns, in an embodiment.

FIG. 2B is a perspective view of a nanofiber yarn electrochemical sensor, in an embodiment.

FIG. 2C is a cross-sectional view of a nanofiber yarn electrochemical sensor in which sensing agents are disposed on a surface of nanofiber yarns, the view taken perpendicular to a longitudinal axis of the nanofiber yarns, in an embodiment.

FIG. 2D is a cross-sectional view of a nanofiber yarn electrochemical sensor in which nanofiber yarns are adhered onto an exterior surface of a substrate, the view taken perpendicular to a longitudinal axis of the nanofiber yarns, in an embodiment.

FIG. 3 is a plan view a nanofiber yarn electrochemical sensor having three nanofiber yarns and configured to detect two different analytes, in an embodiment.

FIG. 4 is a plan view of a double spiral configuration nanofiber yarn electrochemical sensor, in an embodiment.

FIG. 5 is a perspective view of a nanofiber yarn electrochemical sensor adhered onto a human limb, in an embodiment.

FIG. 6A is a perspective view of a nanofiber yarn electrochemical sensor integrated within a garment and disposed on a human limb so as to detect one or more physiological states, in an embodiment.

FIG. 6B is a magnified view of the nanofiber yarn electrochemical sensor integrated within the garment of FIG. 6A, in an embodiment.

FIG. 7 is a photomicrograph of an example forest of nanofibers on a substrate, in an embodiment.

FIG. 8 is a schematic illustration of an example reactor for nanofiber growth, in an embodiment.

FIG. 9 is a schematic illustration of an example multi-layered nanofiber forest having two layers, in an embodiment.

FIG. 10 is an illustration of an example nanofiber sheet, in an embodiment.

The figures depict various embodiments of the present disclosure for purposes of illustration only. Numerous variations, configurations, and other embodiments will be apparent from the following detailed discussion.

### DETAILED DESCRIPTION

### Overview

Embodiments of the present disclosure include nanofiber yarn based sensors that can be used to detect analytes in an electrolyte, whether in a biological or a non-biological application. In one example, a nanofiber yarn based sensor (“nanofiber sensors” or “sensors” for brevity) includes at least two nanofiber yarns that are spaced apart from one another so as to avoid physical contact (that would form an electrical short circuit) with one another. Each nanofiber yarn of the nanofiber sensor includes a sensing region that is in electrical contact with, and integral with, the rest of the corresponding nanofiber yarn. The sensing regions of the at least two nanofibers are treated with complementary sensing agents so that when the sensing regions (and the corresponding sensing agents) are exposed to the analyte to be detected, a change in electrical state develops and can be detected. Examples of electrical responses detected by the nanofiber sensor include a current, and/or a potential difference between the sensing regions of at least two nanofiber yarns. This response is then communicated through one or more of the nanofiber yarns for interpretation by a processor.

In some examples, the at least two nanofibers are disposed on a substrate that can be in contact with, or adhered to, a surface. In this way, the sensor can be conveniently attached, detached, and re-attached (referred to as “reversible attachment”) to a surface to detect an analyte. In some examples, the at least two nanofibers are integrated within a fabric that can be positioned on a surface without using an adhesive. For example, the fabric can be configured (e.g., through the use of elastic fibers) to urge contact between the nanofiber sensor and the surface on which an analyte is disposed. Regardless, when a sensor is integrated within a fabric, physiological conditions in humans or presence of an analyte on a surface can be monitored by merely wearing a garment or placing a fabric onto the surface, respectively.

Regardless of the technique by which the sensor is placed in contact with the surface to be monitored, the use of nanofiber sensors has a number of advantages. One example advantage includes increased durability to mechanical stress and/or chemical attack compared to conventional ISE sensors. For example, some conventional ISE sensors can be temporarily placed onto human skin using conductive inks. The sensors are fabricated by printing electrical contacts and conductive lines on paper used in the application of “temporary tattoos.” This “tattoo type” printed sensor is treated with appropriate sensing agents for the analyte to be detected. Thus fabricated, the ISE sensor is temporarily adhered to human skin. Electrical leads are then connected to the printed electrical contact regions for the exchange of electrical signals between a power source, the sensor, and a processor. The processor then analyzes electrical signals produced by the sensor and can produce a user-readable output regarding a physiologic condition.

These conventional ISE sensors have a number of drawbacks not found in nanofiber yarn sensors of the present disclosure. For example, because nanofiber yarns can be fabricated into continuous lengths as long as a kilometer, there is no need to print electrical contacts that connect a physically distinct conductor (e.g., an insulated copper wire to/from a power source and/or processor) with the sensor. Rather, the nanofiber yarns used in the sensor can be fabricated to be conductive so that a sensing region of the nanofiber yarn is continuous and integral with (i.e., no joint, joinery, or discontinuity between) a conductive portion that connects the sensing region to a power source and/or processor. This improves the durability of the sensor as a whole and simplifies fabrication of the sensor. In some cases, the lack of a discontinuity and separate electrical connection to a contact interface for nanofiber sensors described herein improves sensitivity and reliability of the sensor.

Electrochemical sensors fabricated with nanofiber yarns are also more mechanically durable as a whole than ISE sensors printed with conductive inks. Conductive inks, while somewhat conformable to contours of the surface to which they are attached, are prone to cracking and delamination from the surface to which they are attached. While tattoo type ISE sensors are sometimes reinforced with a filler material, this is still insufficient in many cases to fabricate a mechanically durable sensor. This is problematic because some types of sensors (e.g., amperometric sensors) require a constant cross-sectional size for the two electrodes to maintain consistent resistivity and thus accurately interpret an electrical signal. The advantage of using nanofiber yarns instead of a conductor formed from a printable ink is that size of the yarns does not change over time because the nanofiber yarns are unlikely to crack or break. A nanofiber yarn also has more surface area (due to the plurality of individual nanofibers within a yarn) by which to conduct electricity than a monolithic ink-based conductor.

Tattoo type ISE sensors are also not configured to be attached, detached, and re-attached (“reversibly attached”) to an underlying surface. The adhesive often used to attach the tattoo type ISE sensor can be mechanically stronger than the conductive ink itself, thus leading to mechanical failure of the ISE sensor as the sensor is being detached. Even if a weaker adhesive is used, the mechanical integrity of the ink is generally insufficient for reversible attachment to a surface. Cracking of the conductive ink can degrade the accuracy and performance of the sensor, as described above.

As will be explained below in more detail, embodiments described herein have sufficient mechanical integrity for reversible attachment. As a result, nanofiber yarn sensors are mechanically durable sensors that can be used repeatedly in any number of challenging applications without mechanical failure. Other advantages will be apparent in light of the following description.

### Nanofiber Forests, Sheets, and Yarns

As used herein, the term “nanofiber” means a fiber having a diameter less than 1 μm. While the embodiments herein are primarily described as fabricated from carbon nanotubes, it will be appreciated that other carbon allotropes, whether graphene, micron or nano-scale graphite fibers and/or plates, and even other compositions of nano-scale fibers such as boron nitride may be used to fabricate nanofiber sheets using the techniques described below. As used herein, the terms “nanofiber” and “carbon nanotube” encompass both single walled carbon nanotubes and/or multi-walled carbon nanotubes in which carbon atoms are linked together to form a cylindrical structure. In some embodiments, carbon nanotubes as referenced herein have between 4 and 10 walls. As used herein, a “nanofiber sheet” or simply “sheet” refers to a sheet of nanofibers aligned via a drawing process (as described in PCT Publication No. WO 2007/015710, and incorporated by reference herein in its entirety) so that a longitudinal axis of a nanofiber of the sheet is parallel to a major surface of the sheet, rather than perpendicular to the major surface of the sheet (i.e., in the as-deposited form of the sheet, often referred to as a “forest”).

The dimensions of carbon nanotubes can vary greatly depending on production methods used. For example, the diameter of a carbon nanotube may be from 0.4 nm to 100 nm and its length may range from 10 μm to greater than 55.5 cm. Carbon nanotubes are also capable of having very high aspect ratios (ratio of length to diameter) with some as high as 132,000,000:1 or more. Given the wide range of dimensional possibilities, the properties of carbon nanotubes are highly adjustable, or tunable.

Due to their unique structure, carbon nanotubes possess particular mechanical, electrical, chemical, thermal and optical properties that make them well-suited for certain applications. In particular, carbon nanotubes exhibit superior electrical conductivity, high mechanical strength, good thermal stability and are also hydrophobic. In addition to these properties, carbon nanotubes may also exhibit useful optical properties. For example, carbon nanotubes may be used in light-emitting diodes (LEDs) and photo-detectors to emit or detect light at narrowly selected wavelengths. Carbon nanotubes may also prove useful for photon transport and/or phonon transport.

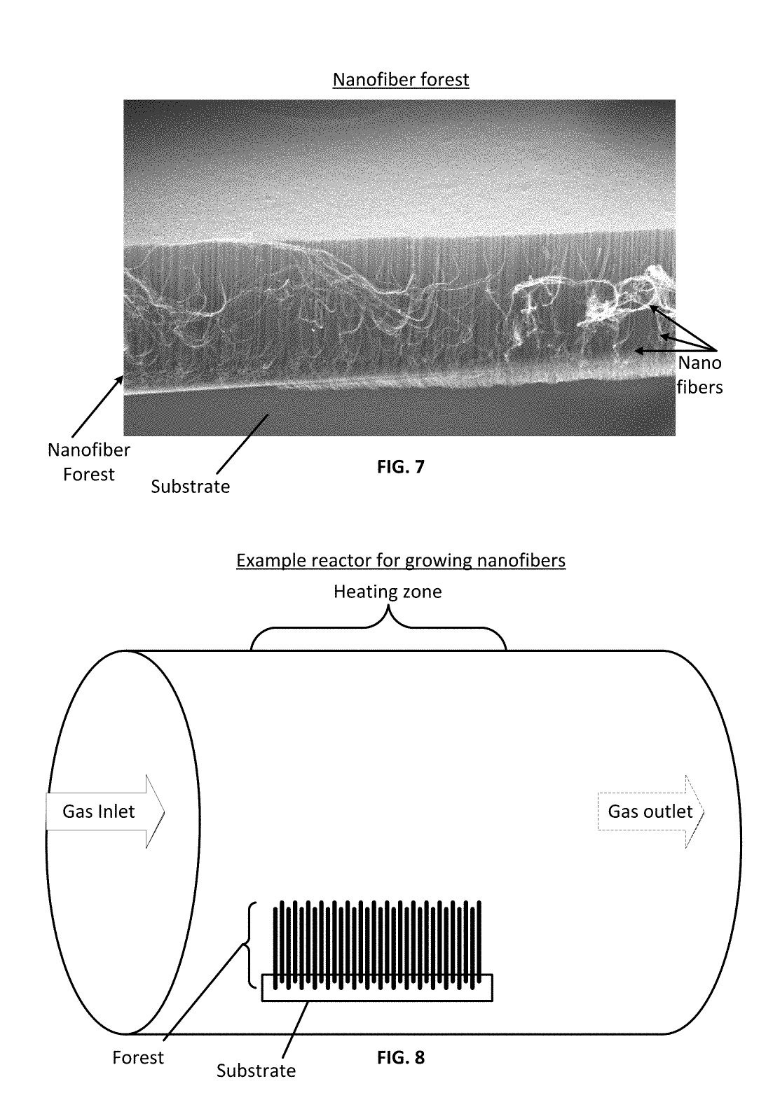

In accordance with various embodiments of the subject disclosure, nanofibers (including but not limited to carbon nanotubes) can be arranged in various configurations, including in a configuration referred to herein as a “forest.” As used herein, a “forest” of nanofibers or carbon nanotubes refers to an array of nanofibers having approximately equivalent dimensions that are arranged substantially parallel to one another on a substrate. FIG. 7 shows an example forest of nanofibers on a substrate. The substrate may be any shape but in some embodiments the substrate has a planar surface on which the forest is assembled. As can be seen in FIG. 7, the nanofibers in the forest may be approximately equal in height and/or diameter.

In some embodiments, the nanofibers of the forest may each be oriented toward the substrate at approximately the same angle. For example, the nanofibers of the forest may be angled between 45° and 135° in relation to the substrate. In particular embodiments, the nanofibers of the forest may be oriented between 75° and 105° from the substrate and in select embodiments the nanofibers may be oriented approximately 90° from the substrate.

Nanofiber forests as disclosed herein may be relatively dense. Specifically, the disclosed nanofiber forests may have a density of at least 1 billion nanofibers/cm². In some specific embodiments, a nanofiber forest as described herein may have a density of between 10 billion/cm² and 30 billion/cm². In other examples, the nanofiber forest as described herein may have a density in the range of 90 billion nanofibers/cm². The forest may include areas of high density or low density and specific areas may be void of nanofibers. The nanofibers within a forest may also exhibit inter-fiber connectivity. For example, neighboring nanofibers within a nanofiber forest may be attracted to one another by van der Waals forces.

Various methods can be used to produce nanofiber forests. For example, in some embodiments nanofibers may be grown in a high-temperature furnace. In some embodiments, catalyst may be deposited on a substrate, placed in a reactor and then may be exposed to a fuel compound that is supplied to the reactor. Substrates can withstand temperatures of greater than 800° C. to 1000° C. and may be inert materials. The substrate may comprise stainless steel or aluminum disposed on an underlying silicon (Si) wafer, although other ceramic substrates may be used in place of the Si wafer (e.g., alumina, zirconia, SiO2, glass ceramics). In examples where the nanofibers of the forest are carbon nanotubes, carbon-based compounds, such as acetylene may be used as fuel compounds. After being introduced to the reactor, the fuel compound(s) may then begin to accumulate on the catalyst and may assemble by growing upward from the substrate to form a forest of nanofibers.

A diagram of an example reactor for nanofiber growth is shown in FIG. 8. As can be seen in FIG. 8, the reactor may include a heating zone where a substrate can be positioned to facilitate nanofiber forest growth. The reactor also may include a gas inlet where fuel compound(s) and carrier gases may be supplied to the reactor and a gas outlet where expended fuel compounds and carrier gases may be released from the reactor. Examples of carrier gases include hydrogen, argon, and helium. These gases, in particular hydrogen, may also be introduced to the reactor to facilitate growth of the nanofiber forest. Additionally, dopants to be incorporated in the nanofibers may be added to the gas stream. Example methods of adding dopants during deposition of the nanofiber forest are described at paragraph 287 of PCT Publication No. WO 2007/015710 and are incorporated by reference herein. Other example methods of doping or providing an additive to the forest include surface coating, dopant injection, or other deposition and/or in situ reactions (e.g., plasma-induced reactions, gas phase reaction, sputtering, chemical vapor deposition). Example additives include polymers (e.g., poly(vinyl alcohol), poly(phenylene tetrapthalamide) type resins, poly(p-phenylene benzobisoxazole), polyacrylonitrile, poly(styrene), poly(ether etherketone) and poly(vinyl pyrrodidone, or derivations and combinations thereof), gases of elements or compounds (e.g., fluorine), diamond, palladium and palladium alloys, among others.

The reaction conditions during nanofiber growth can be altered to adjust the properties of the resulting nanofiber forest. For example, particle size of the catalyst, reaction temperature, gas flow rate and/or the reaction time can be adjusted as needed to produce a nanofiber forest having the desired specifications. In some embodiments, the position of catalyst on the substrate is controlled to form a nanofiber forest having a desired pattern. For example, in some embodiments catalyst is deposited on the substrate in a pattern and the resulting forest grown from the patterned catalyst is similarly patterned. Exemplary catalysts include iron with a buffer layer of silicon oxide (SiO₂) or aluminum oxide (Al₂O₃). These may be deposited on the substrate using chemical vapor deposition (CVD), pressure assisted chemical vapor deposition (PCVD), electron beam (eBeam) deposition, sputtering, atomic layer deposition (ALD), laser assisted CVD, plasma enhanced CVD, thermal evaporation, various electrochemical methods, among others.

In some particular embodiments, multiple nanofiber forests may be sequentially grown on the same substrate to form a multilayered nanofiber forest. For example, a first nanofiber forest is formed on the substrate and a second nanofiber forest is formed on top of the first nanofiber forest with the nanofibers of the second nanofiber forest being aligned approximately end-to-end with the nanofibers of the first nanofiber forest. Multi-layered nanofiber forests may include any number of forests. For example, a multi-layered forest may include two, three, four, five or more forests. Multi-layered nanofiber forests can be formed by numerous suitable methods, such as by forming a first nanofiber forest on the substrate, depositing catalyst on the first nanofiber forest and then introducing additional fuel compound to the reactor to encourage growth of a second nanofiber forest from the catalyst positioned on the first nanofiber forest.

After formation, the nanofiber forest may optionally be modified. For example, in some embodiments, the nanofiber forest may be exposed to a treatment agent such as an oxidizing or reducing agent. In some embodiments, the nanofibers of the forest may optionally be chemically functionalized by a treatment agent. Treatment agent may be introduced to the nanofiber forest by any suitable method, including but not limited to chemical vapor deposition (CVD) or any of the other techniques and additives/dopants presented above. In some embodiments, the nanofiber forest may be modified to form a patterned forest. Patterning of the forest may be accomplished, for example, by selectively removing nanofibers from the forest. Removal can be achieved through chemical or physical means.

In addition to arrangement in a forest configuration, the nanofibers of the subject application may also be arranged in a sheet configuration. As used herein, the term “nanofiber sheet,” “nanotube sheet,” or simply “sheet” refers to an arrangement of nanofibers where the nanofibers are aligned end to end in a plane. In some embodiments, the sheet has a length and/or width that is more than 100 times greater than the thickness of the sheet. In some embodiments, the length, width or both, are more than 10³, 10⁶ or 10⁹ times greater than the average thickness of the sheet. A nanofiber sheet can have a thickness of, for example, between approximately 5 nm and 30 μm and any length and width that are suitable for the intended application. In some embodiments, a nanofiber sheet may have a length of between 1 cm and 10 meters and a width between 1 cm and 1 meter. These lengths are provided merely for illustration. The length and width of a nanofiber sheet are constrained by the configuration of the manufacturing equipment and not by the physical or chemical properties of any of the nanotubes, forest, or nanofiber sheet. For example, continuous processes can produce sheets of any length. These sheets can be wound onto a roll as they are produced.

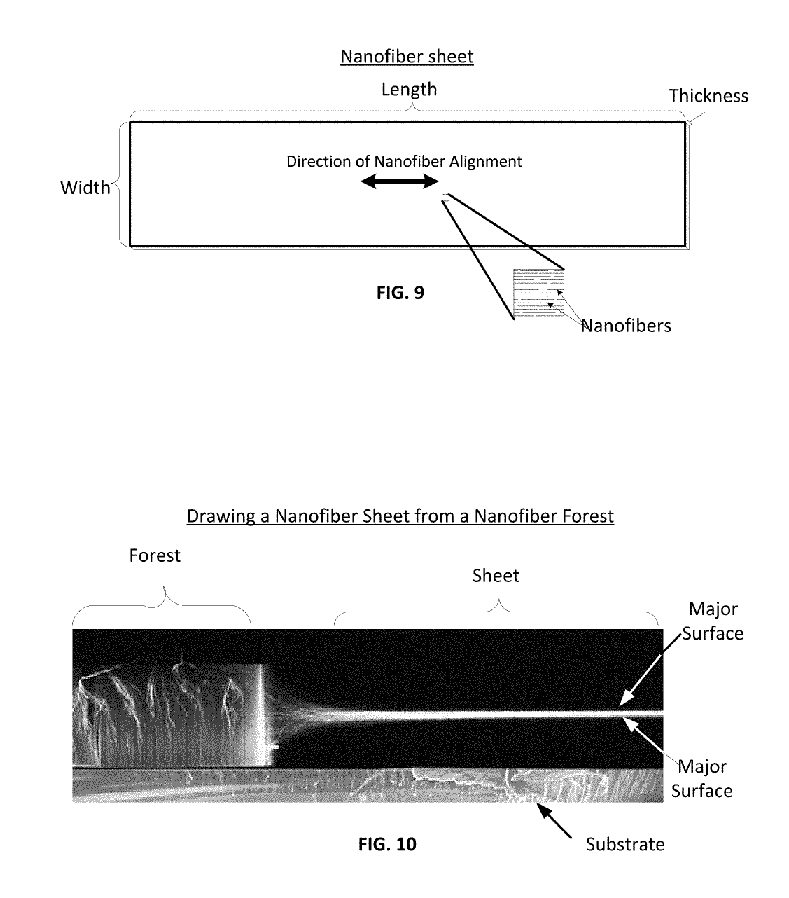

An illustration of an example nanofiber sheet is shown in FIG. 9 with relative dimensions illustrated. As can be seen in FIG. 9, the axis in which the nanofibers are aligned end-to end is referred to as the direction of nanofiber alignment. In some embodiments, the direction of nanofiber alignment may be continuous throughout an entire nanofiber sheet. Nanofibers are not necessarily perfectly parallel to each other and it is understood that the direction of nanofiber alignment is an average or general measure of the direction of alignment of the nanofibers.

Nanofiber sheets may be stacked on top of one another to form a multi-layered sheet stack. Nanofiber sheets may be stacked to have the same direction of nanofiber alignment or to have different directions of nanofiber alignment. Any number of nanofiber sheets may be stacked on top of one another to form a multi-layered nanofiber sheet stack. For example, in some embodiments, a nanofiber sheet stack may include 2, 3, 4, 5, 10, or more individual nanofiber sheets. The direction of nanofiber alignment on adjoining sheets in a stack may differ by less than 1°, less than 5° or less than 10°. In other embodiments, the direction of nanofiber alignment on adjoining or interleaved sheets may differ by more than 40°, more than 45°, more than 60°, more than 80°, or more than 85°. In specific embodiments, the direction of nanofiber alignment on adjoining or interleaved sheets may be 90°. Multi-layer sheet stacks may include other materials such as polymers, metals and adhesives in between individual nonfiber sheets.

Nanofiber sheets may be assembled using any type of suitable process capable of producing the sheet. In some example embodiments, nanofiber sheets may be drawn from a nanofiber forest. An example of a nanofiber sheet being drawn from a nanofiber forest is shown in FIG. 10.

As can be seen in FIG. 10, the nanofibers may be drawn laterally from the forest and then align end-to-end to form a nanofiber sheet. In embodiments where a nanofiber sheet is drawn from a nanofiber forest, the dimensions of the forest may be controlled to form a nanofiber sheet having particular dimensions. For example, the width of the nanofiber sheet may be approximately equal to the width of the nanofiber forest from which the sheet was drawn. Additionally, the length of the sheet can be controlled, for example, by concluding the draw process when the desired sheet length has been achieved.

Nanofiber sheets have many properties that can be exploited for various applications. For example, nanofiber sheets may have tunable opacity, high mechanical strength and flexibility, thermal and electrical conductivity, and may also exhibit hydrophobicity. Given the high degree of alignment of the nanofibers within a sheet, a nanofiber sheet may be extremely thin. In some examples, a nanofiber sheet is on the order of approximately 10 nm thick (as measured within normal measurement tolerances), rendering it nearly two-dimensional. In other examples, the thickness of a nanofiber sheet can be as high as 200 nm or 300 nm. As such, nanofiber sheets may add minimal additional thickness to a component.

As with nanofiber forests, the nanofibers in a nanofibers sheet may be functionalized by a treatment agent by adding chemical groups or elements to a surface of the nanofibers of the sheet and that provide a different chemical activity than the nanofibers alone. Functionalization of a nanofiber sheet can be performed on previously functionalized nanofibers or can be performed on previously unfunctionalized nanofibers. Functionalization can be performed using any of the techniques described herein including, but not limited to CVD, and various doping techniques.

Nanofiber sheets, prior to metallization and/or polymer infiltration, as disclosed herein may also have high purity, wherein more than 90%, more than 95% or more than 99% of the weight percent of the nanofiber sheet is attributable to nanofibers, in some instances. Similarly, the nanofiber sheet may comprise more than 90%, more than 95%, more than 99% or more than 99.9% by weight of carbon.

The nanofiber sheet, thus having been drawn from a forest or otherwise produced, may then be processed in to a yarn (among other configurations). The nanofiber sheet may be “densified” prior to being processed into a yarn by, for example, using a solvent. The solvent can be used to introduce, “infiltrate” the nanofiber sheet with a polymer to broaden the physical conditions in which the nanofiber sheet may be applied.

In other embodiments, the infiltrating polymer itself will densify a nanofiber sheet. Using an infiltrating polymer to densify a nanofiber sheet instead of a separate solvent has a number of benefits. These benefits include reduced cost and improved convenience of fabrication because a separate manufacturing step and additional material are omitted from the process.

The nanofiber sheet can be further processed into a nanofiber yarn, which is described in PCT Application Publication No. WO 2007/015710, filed Nov. 9, 2015 which is incorporated by reference herein in its entirety.

For example, nanofiber yarns used in the sensors of the present disclosure can be fabricated using a “false twist” technique. In a false twist spinning technique, a twist is introduced to an untwisted nanofiber strand (which is merely a nanofiber sheet that may have a width less than the substrate) by twisting the nanofiber strand at points between ends of the strand (i.e., in the “middle” of an untwisted strand). This is in contrast to the “true twist” technique where one end of a strand is fixed and the opposing end of the strand is rotated to introduce the twist to intervening portions of yarn.

In some embodiments, additional materials can be introduced into a nanofiber sheet prior to false twist spinning the nanofiber sheet into a yarn by suspending or dissolving one more additional materials into a densifying fluid and providing the fluid and additional material to the sheet (or strand). The additional material(s) are carried into (also known as “infiltrating” or “imbibing”) the nanofibers and/or the gaps between nanofibers by the fluid provided to the untwisted nanofiber sheet (or strand if the sheet is in the process of being drawn but not yet spun into a yarn). Examples of additional materials include conductive nanoparticles and nanowires (silver (Ag), copper (Cu), gold (Au), combinations thereof), magnetic nanoparticles (iron (Fe), nickel (Ni), neodymium (Nd), combinations thereof), carbon nanotubes and fullerenes, polymers, oligomers, small molecules, among others. In some examples, a degree of densification (as measured by the volume reduction of the nanofiber sheet) is less for an infiltrated sheet than for a fully densified sheet (e.g., a sheet treated with an organic solvent that is later removed, as described below) because some of the free volume between the individual fibers is occupied by the material infiltrated into the sheet even after volatile components of the infiltrated material are removed.

The advantage of adding additional materials to a nanofiber sheet or nanofiber strand via a densifying fluid is that the particles can be moved to an interior of the nanofiber strand (and therefore ultimately disposed within an interior of a nanofiber yarn). Furthermore, a protective material can be introduced into a nanofiber sheet or nanofiber strand via a fluid along with the nanoparticle so that the nanoparticles are protected from environmental, physical, or chemical degradation. An example of a protective material that can be used to inhibit corrosion of some types of nanoparticles (e.g., Ag nanoparticles, Fe nanoparticles) is polydimethylsiloxane (PDMS). The PDMS can be dissolved by a solvent that also suspends, for example, Ag nanowires, both of which are then provided to a nanofiber sheet or nanofiber strand. Thus, the Ag nanofibers are partially or entirely coated by PDMS, thus inhibiting corrosion (commonly referred to as “tarnishing”). This helps preserve the conductivity exhibited by nanofiber yarns that include the Ag nanofibers.

In some examples, as described in U.S. Patent Appl. No. 62/383,017, filed on Sep. 2, 2016 and incorporated by reference herein in its entirety, nanofiber sheets are optionally “metallized.” “Metallizing” refers to a process in which one or more metal layers are conformally deposited or otherwise disposed on outer surfaces of the aligned nanofibers within the nanofiber sheet. The conformal metal layer (or layers) are disposed not only on an outer surface of the sheet as a whole, nor only on the outer surfaces of individual carbon nanofibers that are exposed at the outer surface of the nanofiber sheet. Rather, by selecting an appropriate metal and deposition process, the conformal metal layer penetrates, at least partially, beyond a sheet surface to conform to outer surfaces of nanofibers disposed within the sheet itself as well as on nanofibers at the exposed surface of the nanofiber sheet. This deposition can be performed on an individual sheet(s) that are then optionally stacked or performed on an entire stack of nanofiber sheets. Metallizing a nanofiber sheet prior to densifying the nanofiber sheet is beneficial in some embodiments because the un-densified nanofiber sheet defines greater spaces between fibers, thus enabling a more uniform distribution of metal on the fiber surfaces both at a surface of the nanofiber sheet and within the body of the nanofiber sheet.

Examples of processes used to deposit a metallization layer include, but are not limited to chemical vapor deposition (CVD), pressure assisted chemical vapor deposition (PCVD), electron beam (eBeam) deposition, sputtering, atomic layer deposition (ALD), electroplating, laser assisted CVD, plasma-enhanced CVD, thermal evaporation, electrochemical methods (such as electroplating), among others. In some examples, metallic nanoparticles are deposited (rather than a conformal layer).

In other examples, non-metallic materials may be deposited using the processes described above in the context of metallization. For example, magnesium diboride, semiconductors (e.g., silicon, germanium, II-VI semiconductors, III-V semiconductors), other carbon allotropes (e.g., graphite, diamond, fullerenes), polymers, ceramics (e.g., aluminum oxide, tungsten carbide, silicon dioxide), titanium dioxide, lithium ion phosphate, nanoparticles, nanoflakes, nanowires, among others.

In many cases, carbon complexes, including carbon nanotube sheets, are difficult surfaces on which to adhere metals, particularly for less reactive metals, (e.g., noble metals like gold, silver, copper) due to poor adhesion. To overcome this challenge, a first conformal layer of a carbide-forming metal, such as tungsten, molybdenum, titanium, niobium, among others is first deposited onto the nanotube sheet. Other carbide-forming metals and/or alloys may be used instead of titanium, including iron and zinc, zirconium, hafnium, vanadium, tantalum, chromium, among others. This first conformal layer is, in embodiments any of the following thicknesses: from 1 nm to 10 nm, from 1 nm to 5 nm, from 5 nm to 10 nm, from 2 nm to 8 nm, from 3 nm to 7 nm, from 3 nm to 6 nm, from 6 nm to 9 nm, and from less than 30 nm.

Upon depositing the first conformal layer of a carbide-forming metal, in some examples a second conformal layer is deposited on the first conformal layer. Because the second conformal layer adheres to the first conformal layer, any of a variety of metals and metal alloys may be used including, but not limited to gold, silver, copper, nickel, palladium, aluminum, iron, tin, and alloys thereof. The second conformal layer is, in embodiments, any of the following thicknesses: from 10 nm to 300 nm, from 10 nm to 100 nm, from 10 nm to 200 nm, from 100 nm to 200 nm, from 200 nm to 300 nm, from 150 nm to 250 nm, among others.

One benefit of a conformal metal layer that is disposed on nanofiber surfaces interior to the nanofiber sheet is that many individual nanofiber surfaces are coated with metal. This reduces the resistivity of the sheet because there are many possible conductive pathways throughout the sheet, not only a few conductive pathways proximate to an outer surface of the sheet.

Another benefit of a conformal metal layer is that the conductivity of the sheet is preserved even upon infiltration of the sheet by an insulating polymer (which can be beneficial in some applications). Because many of the electrical contacts created upon metallization of the sheet are above, below or non-planar with the surface, some electrical connections between nanofibers remain even upon infiltration of an electrically insulating polymer into portions of a metallized layer in the sheet. However, due to, for example, surface energy differences between nanofibers and metals, polymers generally, and adhesives specifically, prefer contact with carbon nanotubes over metal. As a result, metallized portions of a nanotube sheet may resist polymer infiltration, thus preserving a conductive pathway into the sheet even though polymer has infiltrated from one major surface to portions of an opposite major surface of the sheet. In this case, the polymer layer is proximate to a second major surface of the nanofiber composite sheet, opposite to the major surface proximate to the metallized portions of the nanotube sheet.

In one example, a combination of a first conformal layer of titanium and a second conformal layer of copper can produce a nanofiber sheet with a resistance of approximately 5 Ohms/square (within normal measurement tolerances). Absent these conformal metal layers, a sheet resistance of a nanofiber sheet can be in a range of from 650 Ohms/square to 1200 Ohms/square. Furthermore, upon spinning the metallized sheet into a nanofiber yarn, the improved electrical properties are maintained and/or are further improved by the addition of a conductive material (e.g., silver nanofibers) via infiltration, as described above. Furthermore, the addition of the metallized layers can improve the adhesion of metals, such as solders or sensing materials (described below). This in turn improves the performance of the nanofibers in a sensor.

### Example Sensor Configuration

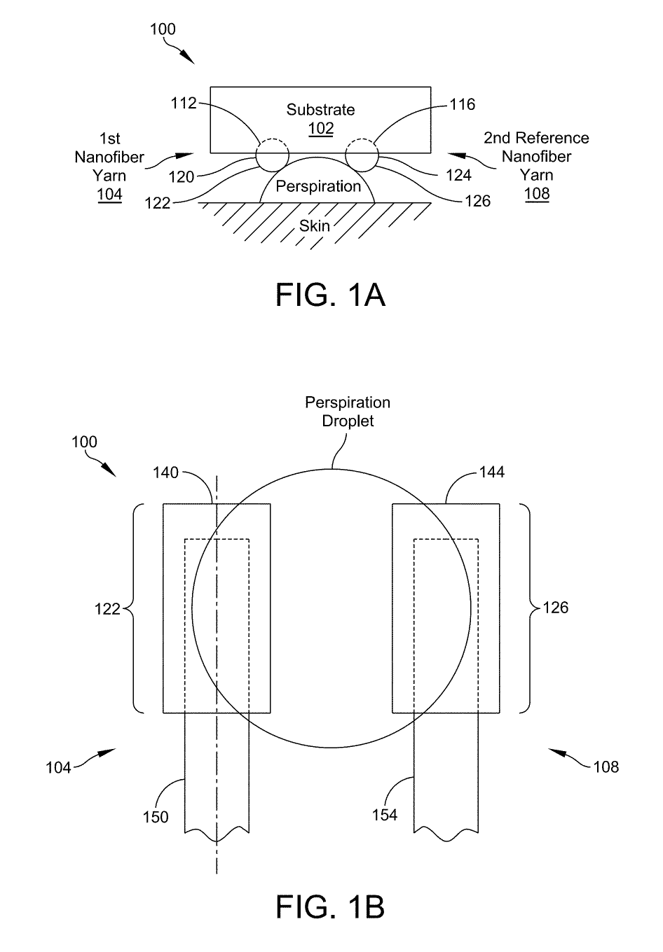

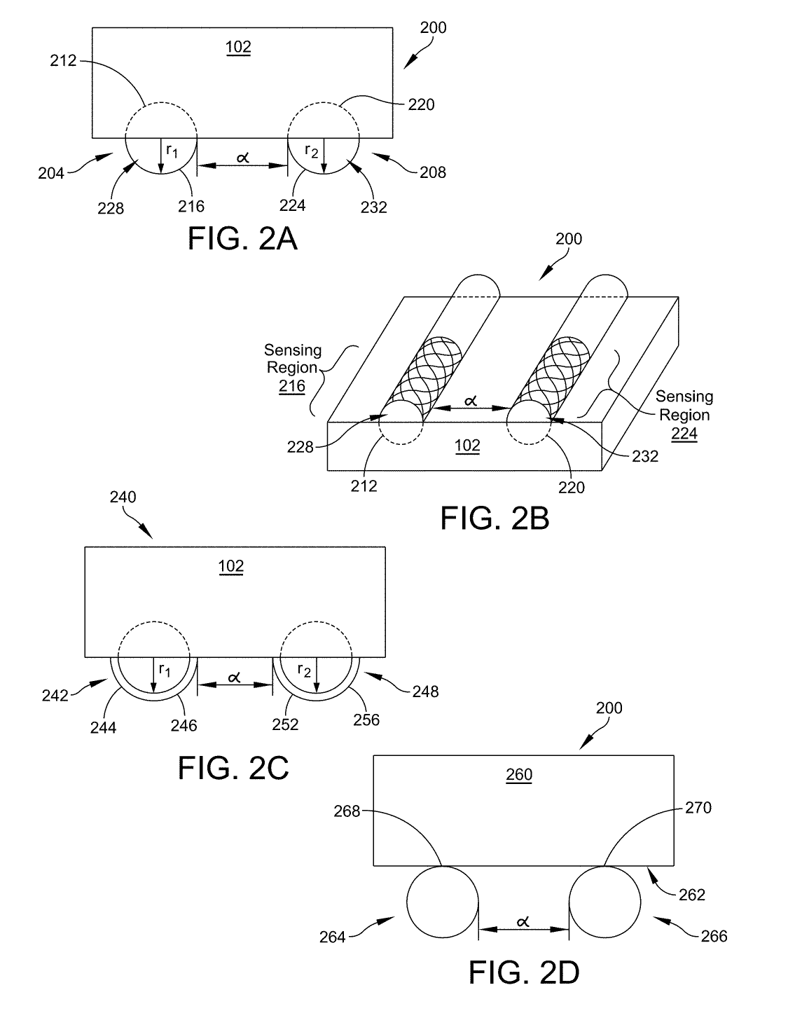

FIG. 1A illustrates a cross-sectional view of an example nanofiber yarn electrochemical sensor 100 (“nanofiber sensor 100”) disposed on perspiring skin, in an embodiment. In this view (and in the views of FIGS. 2A, 2C, and 2D), the cross-section is taken perpendicular to a longitudinal axis of the nanofiber yarns (shown in FIG. 1B). In FIG. 1A, the nanofiber sensor 100 includes a substrate 102, an electrically conductive first nanofiber yarn 104 and an electrically conductive reference nanofiber yarn 108.

The substrate 102 can comprise a polymer backing, a metallic backing with an insulating intermediate layer between the nanofibers and the metallic backing, fabric, and combinations thereof. In some examples, the substrate 102 also includes an adhesive on at least a side that also includes the nanofiber yarns 104, 108. The optional adhesive enables some embodiments of the nanofiber sensor 100 to be adhered to a surface on which an analyte is to be detected, although this can be accomplished in other ways that do not include an adhesive.

The first electrically conductive nanofiber yarn 104 and the electrically conductive reference nanofiber yarn 108 are fabricated according to methods described above. For convenience of explanation, each of the first electrically conductive nanofiber yarn 104 and the electrically conductive reference nanofiber yarn 108 can be considered to have three portions: a substrate connected portion, an exposed portion, and an electrically connective portion.

The electrically connective portion is integral with the other portions and connects the other portions to a power source and a processor without the need for an intermediate electrical joint (e.g., a separate electrical contact for joining an insulated copper wire and the nanofiber together). The electrically connective portion is not visible in FIG. 1A, but rather is depicted and discussed below in the context of FIGS. 1B, 5, and 6.

A substrate connected portion 112 of the first nanofiber yarn 104 and a substrate connected portion 116 of the reference nanofiber yarn 108 are connected to the substrate 102. In the example nanofiber electrochemical sensor 100 shown in FIG. 1A, the substrate connected portion 112 and the substrate connected portion 116 are both embedded within the substrate 102. Embedding these substrate connected portions 112 and 116 within the substrate 102 is one mechanism by which a reversible attachment can be repeatedly made between the example nanofiber electrochemical sensor 100 and a surface (e.g., skin) without loss of mechanical or electrical integrity in the sensor 100. That is, by embedding the substrate connection portions 112, 116 into the substrate, the mechanical connection between the nanofiber yarns 104, 108 and the substrate 102 is strong enough to withstand repeated attachment and detachment from the surface monitored for the analyte. Other mechanisms of attachment between a substrate and elements of the sensor 100 are presented below. Also, while FIG. 1A depicts approximately half of a cross-sectional area of each of the nanofiber yarns 104, 108 embedded into the substrate 102, other embodiments may have more or less of the nanofibers embedded within the substrate 102.

The exposed portion 120 of the first nanofiber yarn 104 and the exposed portion 124 of the reference nanofiber yarn 108 are not encapsulated, covered, or otherwise obscured by the substrate 102 or by an adhesive used to connect the first nanofiber yarn 104 and the reference nanofiber yarn 108 to the substrate 102. Rather, the exposed portions 120, 124 are configured to contact a surface (e.g., skin in the example of FIG. 1A) so as to detect an analyte.

The exposed portions 120, 124 of the first 104 and reference 108 nanofiber yarns correspond, at least in part, to a first sensing region 122 and a reference sensing region 126, respectively. While shown in cross-section in FIG. 1A, the first sensing region 122 and the reference sensing region 126 are shown in plan views in FIGS. 1B, 3, and 4, and in a perspective view in FIG. 2B.

The first sensing region 122 and the reference sensing region 126 are each treated with one of a set of complementary sensing agents. The sensing agents are selected so as to produce an electrochemical response when in electrical contact with a target analyte. In one example, detecting glucose levels in perspiration is accomplished by coating the first sensing region 122 of the first nanofiber yarn 120 with glucose oxidase enzyme and coating the reference sensing region 126 with silver/silver chloride mixture. When both of the sensing agents on the sensing regions 122 and 126 are in electrical contact with one another through the analyte to be detected (in this case glucose) or in electrical contact with one another through an electrolyte that contains the analyte, an electrical potential (i.e., a voltage) develops between the sensing regions 122, 126. This electrical potential is sensed through the electrically conductive first nanofiber yarn and the electrically conductive reference nanofiber yarn by, ultimately, a processor that correlates a magnitude of the electrical potential with a concentration of glucose in the perspiration.

It will be appreciated that many other first and second (or reference) sensing agent combinations are known. In other embodiments, these other sensing agents can be applied to the first and second sensing regions 122, 126 instead of the enzyme and Ag/AgCl described above for detecting glucose, so as to detect other analytes. Substances that can be detected using embodiments described herein, but with different sensing agents, include, but are not limited to sodium, potassium, pH, uric acid, ascorbic acid, trinitrotoluene (TNT), and ammonium.

“Amperometric” sensors, such as the one described above, are one configuration of circuit that can detect a presence of an analyte. Amperometric sensors can be configured to detect, among other analytes, lactate, cholesterol, creatinine, and urea nitrogen. “Potentiometric” sensors operate on essentially the same principle. However, rather than detecting a potential difference between the first nanofiber yarn 104 and the reference nanofiber yarn 108, amperometric sensors are configured to apply a potential difference to the first and reference nanofiber yarns 104, 108 (via a power source) and detect an amount of current flowing between the sensing regions 122, 126 of the nanofiber yarns 104, 108. The magnitude of the current is then correlated with a concentration of an analyte. Potentiometric sensors can be configured to detect, for example, sodium, potassium, and pH.

FIG. 1B is a plan view of the nanofiber electrochemical sensor of FIG. 1A viewed through the substrate 102, in an embodiment. The depiction in FIG. 1B illustrates a physical relationship between the droplet of perspiration containing an analyte, the nanofiber yarns 104, 108, sensing regions 122, 126, and sensing agents 140, 144. The perspiration droplet shown in FIGS. 1A and 1B shows a physical overlap between the droplet of perspiration (which, will be appreciated, can be any analyte-containing electrolyte not limited to perspiration) and the nanofiber yarns. However, it will be appreciated that a minimum contact between an analyte (or analyte containing electrolyte) and the sensing regions 122, 126 to generate a signal is tangent contact.

Also shown in the plan view perspective of FIG. 1B are the sensing agents 140, 144 that, when coated onto exposed portions of nanofiber yarns 104, 108, form sensing regions 122, 126 of the nanofibers 104, 108. These portions are described above and need no further explanation.

FIG. 1B also shows electrically connective portions 150, 154 of the nanofiber yarns 104, 108, respectively, that are untreated with the sensing agents 140, 144 (and thus not part of the sensing regions 122, 126). The electrically connective portions 150, 154, which are integral with other portions of their corresponding nanofiber yarns 104, 108, provide electrical communication between the sensing portions and the conductive portions of the yarn that are typically distal from the sensing portions. These electrically connective portions 150, 154 can be used to sense a potential difference between the sensing regions 122, 126 thus forming a potentiometric sensor. Alternatively, a power supply (not shown) can apply a potential to sensing regions 122, 126, and a resulting current is sensed, thus forming an amperometric sensor. In another embodiment, conductivity of the analyte can be measured at a variety of frequencies applied and sensed via the sensing regions 122, 126, thus forming a conductometric sensor.

The connective portions 150, 154 are also used to transmit the detected voltage or current signal to a processor. The processor interprets the electrical signal and outputs an analyte concentration (or analyte presence indicator) that is a function of the electrical signal detected by the sensing regions 122, 126. Furthermore, in some embodiments, the electrically connective portions 150, 154 of the nanofiber yarns 104, 108 also physically connect the sensing portions 122, 126 to a processor or an interface at the processor (e.g., a pin connector). In some embodiments, there is at most a single electrical joint between the nanofiber yarns 104, 108 and the processor.

As indicated above, one advantage of using nanofiber yarns 104, 108 for the sensing regions 122, 126 and electrically connective portions 150, 154 is that nanofiber yarns are far more durable than conventional technologies. Unlike printed inks, even those inks reinforced with a filler material, nanofiber yarns are unlikely to crack and are highly conformable and pliable. Cracks in conductive elements or sensing elements of a sensor can cause the sensor as a whole to become inoperative due to interruption of electrical signals. Cracks can also cause an incorrect determination of an analyte concentration particularly in amperometric sensors by reducing a cross-sectional area of the conducting portion and thus incorrectly increasing a value of an assumed current. Because of the mechanical and chemical durability of nanofiber yarns, and their ability to be mechanically manipulated, moved, twisted, knotted without a change in electrical resistance, cross-sectional area or cracking, they provide a more durable and more accurate sensor than conventional technologies. These properties can make them useful, for example, in flexible fabrics.

### Yarn, Sensing Agent and Substrate

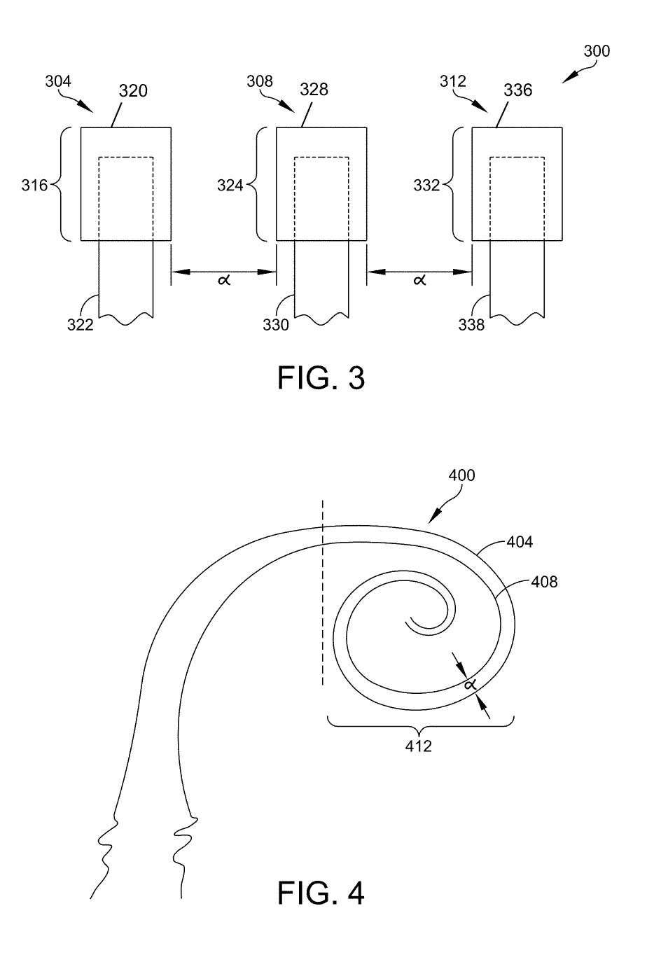

FIGS. 2A, 2B, 2C, 2D, and 3 illustrate alternative views and/or configurations of example nanofiber sensors.

FIG. 2A illustrates an alternative embodiment of a nanofiber sensor 200. The nanofiber sensor 200 includes the substrate 102, a first nanofiber yarn 204 and a reference nanofiber yarn 208.

The first nanofiber yarn 204 has a radius r₁ and the reference nanofiber yarn 208 has a radius r₂. The radii r₁ and r₂ can be of any appropriate value and may be, for example, within any of the following ranges of values: 5 μm to 300 μm; from 5 μm to 200 μm; from 5 μm to 100 μm; from 200 μm to 300 μm; from 100 μm to 200 μm; from 10 μm to 50 μm; from 20 μm to 30 μm; from 50 μm to 100 μm. The radii r₁ and r₂ need not be the same value even though they are depicted as having similar values in FIG. 2A. The radius of a nanofiber yarn may be consistent or may vary along its length.

The first nanofiber yarn 204 and the reference nanofiber yarn 208 can be spaced apart by a dimension α (also referred to herein as a “separation distance”) that can be in any of the following ranges depending on the application in which the nanofiber sensor 200 is to be applied: from 1 μm to 5 μm; from 1 μm to 10 μm; from 1 μm to 20 μm; from 1 μm to 100 μm; from 100 μm to 1 cm; from 0.1 mm to 5 mm; from 0.5 mm to 2 mm; from 1 mm to 2 mm; from 3 mm to 5 mm. In some examples in which the nanofiber sensor 200 is configured to detect analytes in human perspiration, the dimension α can be determined based on average minimum perspiration droplet size. The spacing between the fibers, α, can be consistent or varied along the length of the fibers or along the length of the sensing region.

The first nanofiber yarn 204 includes a substrate connected portion 212 and a sensing region 216. The reference nanofiber yarn 208 includes a substrate connected portion 220 and a sensing region 224. These various elements are analogous to elements described above in the context of FIG. 1A.

In the example nanofiber sensor 200, a first sensing agent 228 is associated with the first nanofiber yarn 204 (i.e., at an exposed surface of the sensing region 216 or within a space defined by the exposed surface). The sensing agent can be disposed as a core in the yarn, can be infused throughout the yarn, can be coated on the surface of the yarn, or combinations thereof. The first sensing agent 228 can be associated with the first nanofiber yarn 204 using any of the techniques described above, including applying the first sensing agent 228 via a densifying fluid. In other examples, the first sensing agent 228 is applied, absorbed, diffused, injected, infiltrated, precipitated on, vacuum deposited, e-beam deposited, or otherwise provided to the first nanofiber yarn 204 so as to be attached at the exposed surface of the sensing region 216 or within an interior of the nanofiber yarn 204 itself. A second sensing agent 232 can be similarly disposed on, at, and/or within a nanofiber yarn 208 that can be, for example, a reference electrode.

FIG. 2B shows a perspective view of the nanofiber sensor 200, including the first nanofiber 204 and the reference nanofiber 208 on the substrate 102. The corresponding sensing regions 216, 224 and first sensing agent 228 and second sensing agent 232 are also indicated. Because FIG. 2B merely illustrates the nanofiber sensor 200 in a perspective view, these various elements need no further description.

FIG. 2C illustrates an alternative nanofiber yarn sensor 240 in which the sensing agents are disposed on a surface of their respective yarns. As shown, an electrically conductive first nanofiber yarn 242 has a sensing region 244 that has a sensing agent 246 disposed on the previously exposed surface of the sensing region 244. Similarly, an electrically conductive reference nanofiber yarn 248 has a sensing region 252 that has a sensing agent 256 disposed on the previously exposed surface of the sensing region 252. The sensing agents 246, 256 can be disposed on the exposed surfaces of their corresponding sensing regions 244, 252 by dipping, spraying, wicking, painting, electroplating, vacuum depositing, e-beam depositing or otherwise applying the sensing agents 246, 256 to their respective nanofiber yarns 242, 248.

Any of the sensing agents 228, 232, 246, 256 can be liquid, solid, gel, particulate, or any other phase of matter, or combination of phases of matter. In some cases, one or more precursors of sensing agents are infiltrated or otherwise provided to a corresponding nanofiber 204, 208, 242, 248 and reacted in situ so as to form on or within (or both) their respective nanofibers 204, 208, 242, 248.

FIG. 2D illustrates alternative nanofiber yarn sensor 258 in which nanofibers 264 and 266 are connected to an outer surface 262 of a substrate 260. This connection is illustrated in FIG. 2D at connections 268 and 270 which are areas of contact between the nanofibers 264, 266 and the outer surface 262 of the substrate 260. The example nanofiber yarn sensor 258 is in contrast to embodiments described above in which the nanofibers are partially embedded within a substrate 102 itself. The nanofiber yarns and the substrate 260 can exhibit various surface energies. Higher surface energies can facilitate analyte detection by helping to spread water droplets. Lower surface energies may be useful when a buildup of aqueous fluid is helpful prior to any analytical measurements. The yarns and/or substrates can exhibit a water contact angle of, for example, less than 45°, less than 70°, less than 90°, less than 120°, greater than 60°, greater than 90°, greater than 120° or greater than 145°.

The connection between the nanofibers 264, 266 and the outer surface 262 can be accomplished using an adhesive or mechanical attachment (e.g., thread, conventional or nanofiber yarn, staples) that does not inhibit contact between an analyte and the nanofibers 264, 266. In some cases, the yarns can be retained on a surface using only van der Waals forces and will not require adhesives or connectors. Yarns may also be embedded or partially embedded in a surface by softening or melting the surface and pressing the yarns into the surface. This may be particularly application with a polymer surface, such as a thermoplastic polymer. In other embodiments, outer surface 262 can include the hook half of a hook and loop fastener. The yarns can then be retained on the surface by the hooks which are capable of grabbing the yarns.

### Alternative Sensor Configurations

FIGS. 3 and 4 illustrate alternative configurations of various nanofiber yarn sensors. FIG. 3 illustrates a sensor 300 that includes a first nanofiber yarn 304, a reference nanofiber yarn 308, and additional nanofiber yarn 312. The first nanofiber yarn 304 includes a sensing region 316, a sensing agent 320, and an electrically connective portion 322. The reference nanofiber yarn 308 includes a reference sensing region 324, a sensing agent 328, and an electrically connective portion 330. The additional nanofiber yarn 312 includes an additional sensing region 332, an additional sensing agent 336, and an electrically connective portion 338. The various elements individually can be any one or more of the embodiments described above. For example, while sensing agents 320, 328, 336 are shown as disposed on a surface of their corresponding nanofiber yarns 302, 308, 312, other embodiments may include sensing agents 320, 328, 336 disposed, in whole or in part, within an interior of the respective nanofiber yarns 302, 308, 312. Any of the configurations of the nanofiber yarns 302, 308, 312 with respect to a substrate (or fabric) described herein may also be applied to the sensor 300. A three sensor configuration can be used, for example, to sense two different properties simultaneously. In other configurations, a three sensor system can sense a single property or analyte but can be redundant or may provide an average reading or may be used for simultaneous double sensing to reduce false positive readings. In other cases, the two sensors may test for the same property or analyte but may cover different ranges.

The sensing agents 320, 328, 336 can be selected so that two different analytes can be detected. That is, the sensing agent 328 on the reference nanofiber yarn 308 is selected to be a reference for both of sensing agent 320 and sensing agent 336 even though the sensing agent 320 and sensing agent 336 are selected to detect two chemically different analytes. In one example, the sensing agent 328 on the reference nanofiber yarn 308 can be AgCl. The AgCl acts as an electrochemical reference for glucose oxidase and lactate oxidase, which can be used as the sensing agents 320 and 336, respectively

While only three nanofiber yarns 302, 308, 312 with corresponding sensing agents 320, 328, 336 are shown in FIG. 3, it will be appreciated that other nanofiber yarn sensors can embodiment a plurality of nanofiber yarns to detect a presence of multiple different analytes, whether using one reference nanofiber yarn 308 or multiple reference nanofiber yarns. This is in part because the nanofiber yarns can be a small as 10 mm to 30 mm is diameter and so many nanofiber yarns can be integrated on a substrate (or within a fabric) without inconveniencing the user. For example, a single nanofiber yarn sensor could be configured to detect concentration of glucose, potassium, lactase, sodium, and detect pH in perspiration.

FIG. 4 illustrates a double spiral configuration of a nanofiber yarn sensor 400 that can increase a linear length of a sensing region without increasing an areal footprint of the sensor 400. In this embodiment, a first nanofiber yarn 404 and a reference nanofiber yarn 408 are placed apart a distance a as described above within a sensing region 412. Because of the double spiral configuration, the sensing region 412 can occupy a smaller area than is occupied by linearly configured orientations presented above. This can provide for greater precision or a lower limit of detection in a fixed area when compared to a linear configuration.

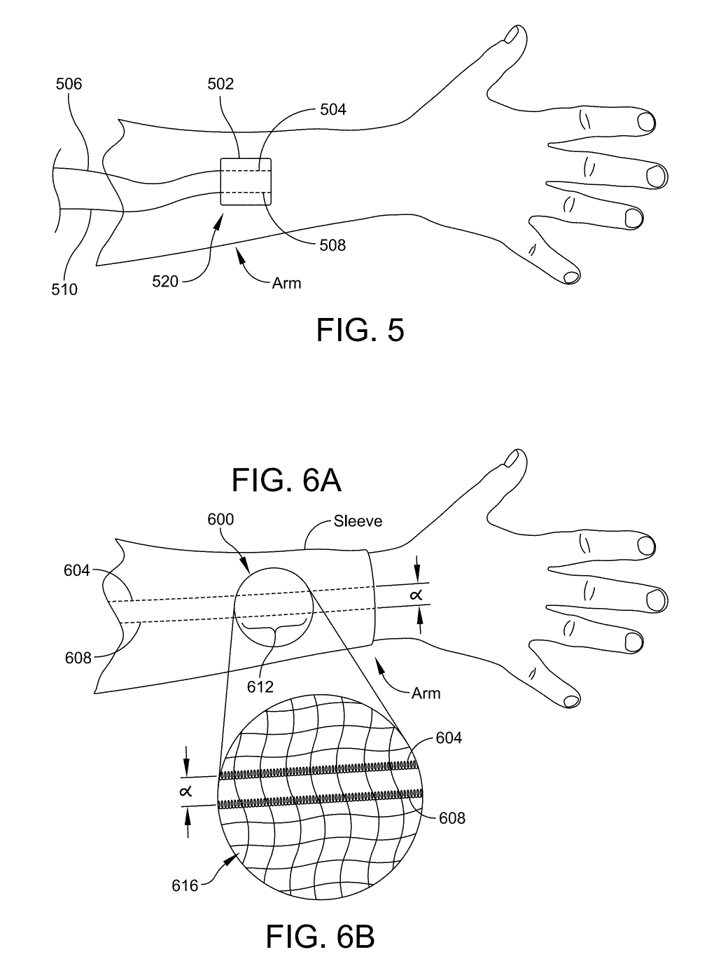

Variations described above in the context of FIGS. 1A, 1B, 2A-2D, 3, and as are described below in FIGS. 5A and 5B are applicable to FIG. 4.

### Sensor Applications

FIG. 5 illustrates a nanofiber sensor 520 of the present disclosure (for example, illustrated by any of example embodiment nanofiber sensors 100, 200, 240, 258, 300, 400) configured as a patch that can be reversibly attached to an underlying surface so as to detect the presence and/or concentration of an analyte thereon. In this illustration, a nanofiber sensor 520 is attached to a substrate 502. Connected to the substrate are a first nanofiber yarn sensing region 504 and a reference nanofiber yarn sensing region 508, as described above. Electrically connective portions 506 and 510 are connected to the sensing regions 504, 508 so as to make electrical connection between the sensing regions and a processor and power source.

In this example, the substrate 502 includes an adhesive (e.g., a medical grade acrylic-based adhesive) that allows reversible attachment to and from skin. A release layer (not shown) can be disposed on the adhesive to prevent accumulation of debris on the adhesive when not attached to skin.

FIGS. 6A and 6B illustrate an example nanofiber yarn sensor 600 that is integrated into a conventional textile or woven or non-woven fabric (whether cotton, wool, linen, synthetic fiber, or a blend thereof). For instance, the nanofiber yarn can be woven into a woven fabric. The conventional textile and the example nanofiber yarn sensor 600 are configured so as to maintain sufficient contact with an underlying surface (in the image depicted, skin of an arm) for detection of an analyte.

FIG. 6A shows the nanofiber yarn sensor 600, which includes a first nanofiber yarn 604, a reference nanofiber yarn 608, a sensing region 612 of the sensor (which includes individual sensing regions corresponding to each of the yarns 604, 608). The yarns 604, 608 within the sensing region 612 are spaced apart by a distance a as described above. As in the preceding examples, the nanofiber yarns 604, 608 each have an electrically connective portion not within the sensing region 612 that is used to make electrical contact between the sensing region of each nanofiber yarn 604, 608 and a processor and/or power source.

FIG. 6B is a magnified view of the example nanofiber yarn sensor 600 shown in FIG. 6A. FIG. 6B illustrates how the nanofiber yarns can be integrated into a fabric 616 itself. The fabric 616 can include elastic fibers that can conform to an underlying surface, thus maintaining contact between the sensing region 612 and the underlying surface. This in turn, facilitates detection of an analyte on the underlying surface by the sensing region, as described above.

### SUMMARY

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A sensor comprising:
a substrate;
an electrically conductive first nanofiber yarn having a first substrate connected portion connected to the substrate and a first exposed portion, the first exposed portion including a first sensing region;
a first sensing agent in contact with the first sensing region of the first nanofiber yarn;
an electrically conductive reference nanofiber yarn having a second substrate connected portion connected to the substrate and a second exposed portion, the second exposed portion including a reference sensing region; and
a second sensing agent in contact with the reference sensing region of the reference nanofiber yarn,
wherein the first sensing agent and the second sensing agent are selected to generate an electrical potential when both the first sensing agent and the second sensing agent are in electrical contact with each other via a first analyte.

2. The sensor of claim 1, wherein the first substrate connected portion and the second substrate connected portion are embedded in the substrate.

3. The sensor of claim 1, wherein the first substrate connected portion and the second substrate connected portion are adhered to an outer surface of the substrate.

4. The sensor of claim 1, wherein the first sensing agent and the second sensing agent are disposed at a surface of the first nanofiber yarn and the reference nanofiber yarn, respectively.

5. The sensor of claim 1, wherein the first sensing agent and the second sensing agent are disposed at least in part within a first interior and a second interior of the first nanofiber yarn and the reference nanofiber yarn, respectively.

6. The sensor of claim 1, wherein a separation distance between the first sensing region and the reference sensing region is from 0.5 mm to 2 mm.

7. The sensor of claim 1, wherein the first sensing region and the reference sensing region are configured as a double spiral.

8. The sensor of claim 1, wherein the substrate is a reversibly attachable adhesive substrate.

9. The sensor of claim 1 integrated into a fabric.

10. The sensor of claim 1, further comprising:
a processor in electrical communication with the electrically conductive first nanofiber yarn and the electrically reference conductive nanofiber yarn; and
a power source connected to the processor and directly connected to the first sensing region and the reference sensing region via a first electrically connective portion of the first nanofiber yarn integral with the first sensing region and a second electrically connective portion of the reference nanofiber yarn integral with the reference sensing region.

11. The sensor of claim 1, further comprising an electrically conductive additional nanofiber yarn comprising:
an additional substrate connected portion connected to the substrate;
an additional exposed portion including an additional sensing region; and
an additional sensing agent different from the first sensing agent and the reference sensing agent,
wherein the additional sensing agent is selected to generate an electrical potential when both the additional sensing agent and the second sensing agent are in contact with an additional analyte different from the first analyte.

12. A garment comprising:
a fabric comprising a plurality of non-conductive threads;
an electrically conductive first nanofiber yarn woven into the fabric with the plurality of non-conductive threads, the first nanofiber yarn having a first sensing region in contact with a first sensing agent; and
an electrically conductive reference nanofiber yarn woven into the fabric with the plurality of non-conductive threads and the first nanofiber yarn, the reference nanofiber yarn having a reference sensing region in contact with a second sensing agent,
wherein the first sensing agent and the second sensing agent are selected to generate an electrical potential when both the first sensing agent and the second sensing agent are in electrical contact with each other via an analyte.

13. The garment of claim 12, wherein a separation distance between the first sensing region and the reference sensing region is from 0.5 mm to 2 mm.

14. The garment of claim 13, wherein the plurality of non-conductive threads urge the first sensing region and the reference sensing region into contact with a surface around which the fabric is disposed.

15. The garment of claim 12, further comprising at least a power source electrically connected to the first nanofiber yarn and the reference nanofiber yarn.

16. The garment of claim 15, wherein:
the electrically conductive first nanofiber yarn further comprises a first electrically connective portion integral with the first sensing region and directly connected to the power source; and
the electrically conductive reference nanofiber yarn further comprises a second electrically connective portion integral with the first sensing region and directly connected to a power source.

17. The garment of claim 12, wherein the first sensing agent and the second sensing agent are disposed at a surface of the first nanofiber yarn and the reference nanofiber yarn, respectively.

18. The garment of claim 12, wherein the first sensing agent and the second sensing agent are disposed at least in part within a first interior and a second interior the first nanofiber yarn and the reference nanofiber yarn, respectively.

19. The garment of claim 12, further comprising a processor in electrical communication with the first electrically conductive nanofiber yarn and the electrically conductive reference nanofiber yarn.
